(19) United States
(12) Patent Application Publication
(10) Pub. No.: US 2015/0150736 A1
(43) Pub. Date: Jun. 4, 2015

(54) TABLETOP LAP/GAUZE COUNTER SYSTEM

(71) Applicants: Marie Pierre, Valley Stream, NY (US); Janice Santiago, Morganville, NJ (US)

(72) Inventors: Marie Pierre, Valley Stream, NY (US); Janice Santiago, Morganville, NJ (US)

(21) Appl. No.: 14/556,209

(22) Filed: Nov. 30, 2014

Related U.S. Application Data
(60) Provisional application No. 61/910,862, filed on Dec. 2, 2013.

(51) Int. Cl. A61F 15/00

(52) U.S. Cl. CPC A61F 15/001

(57) ABSTRACT

A receptacle for storing and counting surgical pads system includes a receptacle for storing and counting surgical pads assembly including a cylindrical receptacle having a flat base, a side wall having a cylindrical rim around a perimeter, and a convex top section having a plurality of inner fins. The receptacle for storing and counting surgical pads system includes the receptacle for storing and counting surgical pads assembly. The receptacle for storing and counting surgical pads assembly houses both used and unused said surgical pads suitably used in a variety of medical and surgical procedures. The receptacle for storing and counting surgical pads assembly is structured and arranged to provide a means for handling, disposing, and keeping track of each said surgical pad effectively ensuring that the surgical pad is not left inside of a user-patient that would potentially cause ill-effects.

### CROSS-REFERENCE TO RELATED APPLICATION

The present application is related to and claims priority from prior provisional application Ser. No. 61/910,862, filed Dec. 2, 2013 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of receptacle devices and more specifically relates to a receptacle for storing and counting surgical pads system.

2. Description of the Related Art

Gauze is popularly thought of as a thin, translucent fabric with a loose open weave. Technically “gauze” is a weave structure in which the warp yarns are arranged in pairs and are crossed before and after each weft yarn keeping the weft firmly in place. This weave structure is used to add stability to fabric, which is important when using fine yarns loosely spaced. However, this weave structure can be used with any weight of yarn, and can be seen in some rustic textiles made from coarse hand-spun plant fiber yarns.

Health care management uses gauze sponges to clean wounds. The materials used in the manufacturing of gauze sponges for medical purposes include cotton and non-woven materials. In addition to its many sizes, plys, and fabrics, gauze sponges can also be sterile and non-sterile. The open weave design of gauze sponges assist with the removal of dead tissue from the skin surface as well as vertically wick fluid from the wound onto any secondary dressing to assist with preventing maceration of skin tissue. In order to avoid leaving gauze sponges in bodies, some kinds of gauze sponge are woven with X-ray detectable thread; however many times gauze may be left in the patient which causes potential injury to the host.

Various attempts have been made to solve problems found in dispensing receptacles that provide a means of counting surgical sponges and gauze art. Among these are found in: U.S. Pat. No. 4,832,198 to Raza Alikhan; U.S. Pat. No. 4,903,837 to Leonard E. Duello; and U.S. Pat. No. 3,948,390 to John G. Ferreri. This prior art is representative of dispensing receptacles. None of the above inventions and patents, taken either singly or in combination, is seen to describe the invention as claimed.

Ideally, a receptacle for storing and counting surgical pads system should be user-friendly and safe in-use and, yet may operate reliably and be manufactured at a modest expense. Thus, a need exists for a receptacle for storing and counting surgical pads system which is structured and arranged to provide a means for handling, disposing, and keeping track of each surgical pad effectively ensuring that the surgical pad is not left inside of a user-patient that would potentially cause ill-effects and to avoid the above mentioned problems.

### BRIEF SUMMARY OF THE INVENTION

In view of the foregoing disadvantages inherent in the known receptacle device art, the present invention provides receptacle for storing and counting surgical pads system (entitled tabletop lap/gauze counter system). The general purpose of the present invention, which will be described subsequently in greater detail is to provide receptacle for storing and counting surgical pads system which is structured and arranged to provide a means for handling, disposing, and keeping track of each surgical pad effectively ensuring that the surgical pad is not left inside of a user-patient (host) that may potentially cause ill-effects.

A receptacle for storing and counting surgical pads system is disclosed herein in a preferred embodiment comprising a receptacle for storing and counting surgical pads assembly including a cylindrical receptacle having a flat base, a side wall having a cylindrical rim around a perimeter, and a convex top section having a plurality of inner fins. The receptacle for storing and counting surgical pads system comprises the receptacle for storing and counting surgical pads assembly. The flat base, the side wall, and the convex top section comprise the cylindrical receptacle. The receptacle for storing and counting surgical pads assembly houses both used and unused surgical pads suitably used in a variety of medical and surgical procedures. The surgical pad may for example comprise a gauze pad. The flat base is able to be placed flat on a planar surface when in an in-use condition. The planar surface comprises a surgical table or the like.

The receptacle for storing and counting surgical pads assembly is structured and arranged to provide a means for handling, disposing, and keeping track of each surgical pad effectively ensuring that the surgical pad is not left inside of a user-patient that would potentially cause ill-effects. The plurality of inner fins comprise turbine-shaped-arcuate fins extruding from a center radius. The turbine-shaped-arcuate fins are structured and arranged to provide separate adjacent compartments for storing a plurality of the surgical pads. The cylindrical rim around the perimeter comprises a lip in preferred embodiments. The lip prevents the surgical pad having at least one bodily fluid from escaping from the cylindrical receptacle thereby preventing potential contamination and excess contact with the at least one bodily fluid.

The cylindrical rim further comprises a plurality of numbers corresponding to each compartment separated by the turbine-shaped-arcuate fins. The plurality of numbers range from 1 through 15 to accommodate up to 15 separate compartments for independently housing up to 15 said surgical pads. The plurality of inner fins comprises 15 to match each separate compartment and the corresponding number located on the cylindrical rim. The plurality of inner fins comprises a different color as a color-coding means to signal to a medical professional that the surgical pad, if the inner fin is visible, has not been replaced and is still inside of the user-patient (a visible indication/warning means).

The plurality of inner fins comprise a concave profile relative to the center radius and the cylindrical rim having the corresponding number. The plurality of inner fins are dimensioned to be pie-shaped so as to fit inside the cylindrical receptacle while accommodating the at least one surgical pad. The receptacle for storing and counting surgical pads assembly may further comprise a pull-string cord for rotating the cylindrical receptacle to a different separate compartment. The pull-string cord comprises a finger-engageable ring to facilitate rotating of the cylindrical receptacle.

A kit is also embodied herein for the receptacle for storing and counting surgical pads system comprising a plurality of the receptacle for storing and counting surgical pads assembly in different sizes; a plurality of the surgical pads including gauze pads and surgical sponges; and a set of user-instructions.

A method of using a receptacle for storing and counting surgical pads system is also disclosed herein comprising the steps of: choosing a receptacle for storing and counting surgical pads assembly in a user-preferred size; placing a receptacle for storing and counting surgical pads assembly on a surgical table; inserting a plurality of unused surgical pads into separate numbered compartments; removing the surgical pad(s) as needed during a medical procedure including surgery; using the surgical pad to absorb bodily fluids of a patient undergoing the medical procedure; placing the used surgical pad, once removed, back into the separate numbered compartment; inventorying both the plurality of used surgical pads and the plurality of unused surgical pads once the medical procedure is complete to make sure all surgical pads are accounted for; disposing of all used surgical pads in a receptacle; removing un-used surgical pads; and cleaning the receptacle for storing and counting surgical pads assembly for future use.

The present invention holds significant improvements and serves as a receptacle for storing and counting surgical pads system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention receptacle for storing and counting surgical pads system (entitled tabletop lap/gauze counter system) constructed and operative according to the teachings of the present invention.

FIG. 1 shows a front perspective view illustrating a receptacle for storing and counting surgical pads system in an ‘in-use’ condition according to an embodiment of the present invention.

FIG. 2 shows a back perspective view illustrating a receptacle for storing and counting surgical pads having 15 different slots of the receptacle for storing and counting surgical pads system according to an embodiment of the present invention.

FIG. 3 shows a back perspective view illustrating a receptacle for storing and counting surgical pads having 30 different slots of the receptacle for storing and counting surgical pads system according to an embodiment of the present invention.

FIG. 4 is a perspective view illustrating a kit of receptacle for storing and counting surgical pads system according to an embodiment of the present invention.

FIG. 5 is a flowchart illustrating a method of use for the receptacle for storing and counting surgical pads system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to receptacle device and more particularly receptacle for storing and counting surgical pads system (entitled tabletop lap/gauze counter system) which is structured and arranged to provide a means for handling, disposing, and keeping track of each surgical pad effectively ensuring that the surgical pad is not left inside of a user-patient that would potentially cause ill-effects.

Generally speaking, the tabletop lap/gauze counter system comprises a specially designed a multi-compartmented, tabletop receptacle that is specially designed to house the soiled gauze pads and sponges used in a variety of medical and surgical procedures.

In this manner, medical professionals are able to possess a neater, safer, and more organized system for disposing of these pads. Featuring a distinct oval shape, this product may be sized appropriately to easily fit onto the surface of any operating or delivery room table, without encumbering medical personnel or the patient. The interior of the Tabletop Lap/Gauze Counter System may include thirty (30) recessed niches, possibly in pie shapes, that serve to house and keep count of gauze pads or surgical sponges. More or less niches may be used.

As such, the unit may be prefilled prior to the procedure with new gauze; as pads are needed, they may be retrieved from the unit and utilized accordingly. When soiled, the pad is replaced in its receptacle, allowing users to put the pad out of the way while also being able to keep a visual inventory of the used pads. To facilitate the latter, each niche may be infused with a brilliant color that, if visible, would signify that a pad has not been replaced and has possibly been left somewhere in the operating room. More importantly, the construction and use of the Tabletop Lap/Gauze Counter may immediately inform medical professionals that a pad may have been inadvertently left inside the patient undergoing the procedure. Unfortunately, this is a common occurrence; medical studies estimate that as many as 1,500 yearly surgeries result in this mishap, with more incidences in Cesarean sections than any other surgery. A dangerous accident, gauze left inside a patient can result in serious, life-threatening infections as well as organ erosion. Thus, a product such as the Lap/Gauze Counter System may prove an essential accessory in any operating room.

Indeed, use of the Tabletop Lap/Gauze Counter System may offer both patients and medical personnel several significant benefits and advantages. Foremost, this novel product may offer a vital component in maintaining quality patient care. Creatively designed to both house and keep track of used gauze pads as well as sponges, the Tabletop Lap/Gauze Counter System may effectively eliminate the possibility of leaving behind contaminated units in sterile operating rooms and, most importantly, the risk of stitching up a patient with the pad inside his or her body. With only a glance at the Tabletop Lap/Gauze Counter, medical personnel may be able to ensure that such a nightmare scenario does not occur, preventing health complications for the patient as well as costly lawsuits that may be leveled against the medical facility.

The construction of the Tabletop Lap/Gauze Counter may offer an additional advantage. Relatively compact, this product may be conveniently placed on the procedure table itself, handily within reach and vision. As such, the Tabletop Lap/Gauze Counter System may eliminate the need to employ the current method of used gauze placement, which involves suspending the pads and sponges from a pole-based stand similar to the type employed for IV bags. Not only are these stands cumbersome to control, but their protruding “arms” and “feet” can present a dangerous tripping hazard in busy operating rooms. With Tabletop Lap/Gauze Counter System at work, doctors and nurses would be able to move freely, without worry.

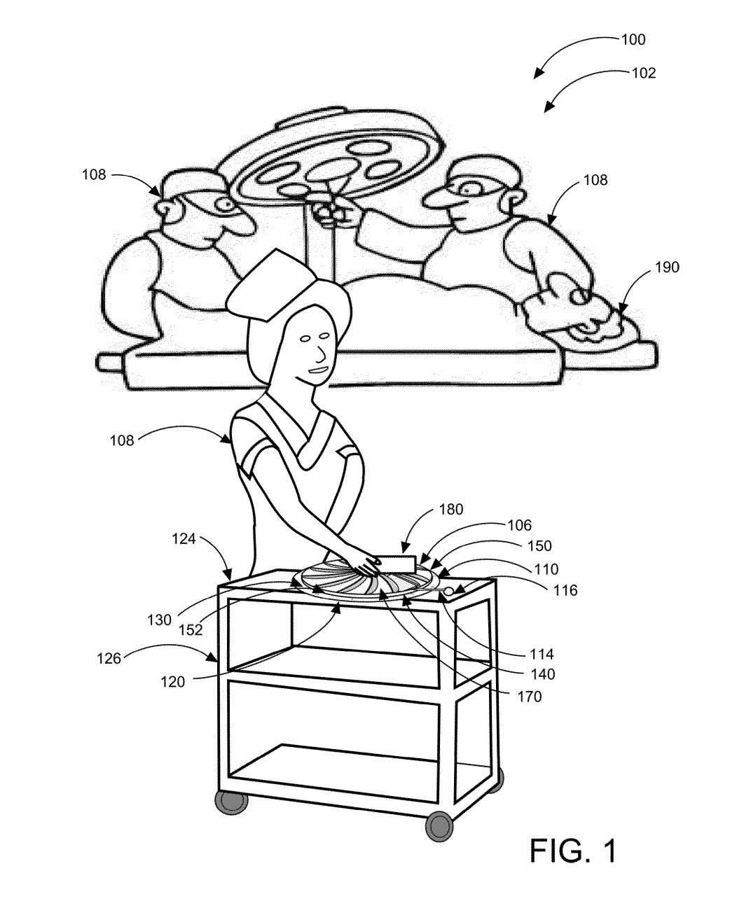

Referring now to the drawings by numerals of reference there is shown in FIGS. 1-3 perspective views illustrating invention receptacle for storing and counting surgical pads assembly 102 of invention receptacle for storing and counting surgical pads system 100 according to an embodiment of the present invention.

Receptacle for storing and counting surgical pads system 100 in a preferred embodiment comprises receptacle for storing and counting surgical pads assembly 102 including cylindrical receptacle 110 having flat base 120, side wall 130 having cylindrical rim 140 around perimeter 150, and convex top section 160 having plurality of inner fins 170. Receptacle for storing and counting surgical pads system 100 comprises receptacle for storing and counting surgical pads assembly 102. Flat base 120, side wall 130, and convex top section 160 comprise cylindrical receptacle 110. Receptacle for storing and counting surgical pads assembly 102 houses both used and unused surgical pads 180 suitably used in a variety of medical and surgical procedures. Surgical pads 180 may comprise a gauze pad 182 or surgical sponge 184. Upon reading this specification, it should be appreciated that, under appropriate circumstances, considering such issues as user preferences, design preference, structural requirements, marketing preferences, cost, available materials, technological advances, etc., other surgical pad arrangements such as, for example, surgical sponges, etc., may be sufficient

Flat base 120 is able to be placed flat on planar surface 124 when in in-use condition 106 as shown in FIG. 1. Planar surface 124 may comprises surgical table 126 as shown in in-use condition 106 of FIG. 1. Receptacle for storing and counting surgical pads assembly 102 is structured and arranged to provide a means for handling, disposing, and keeping track of each surgical pad 180 effectively ensuring that surgical pad 180 is not left inside of a user-patient 190 that would potentially cause ill-effects as shown in in-use condition of FIG. 1. Plurality of inner fins 170 comprise turbine-shaped-arcuate fins 174 extruding from center radius 104. Turbine-shaped-arcuate fins 174 are structured and arranged to provide separate adjacent compartments for storing plurality of surgical pads 180. Cylindrical rim 140 around perimeter comprises lip 152. Lip 152 preferably prevents surgical pad 180 having at least one bodily fluid from escaping from cylindrical receptacle 110 thereby preventing potential contamination and excess contact with the at least one bodily fluid.

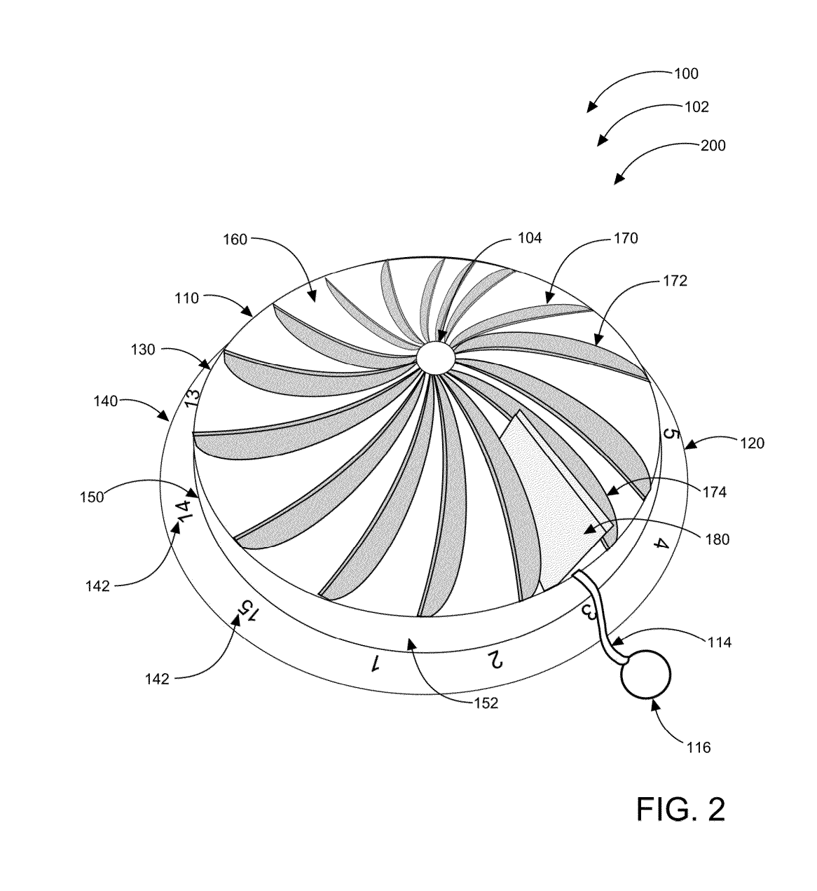

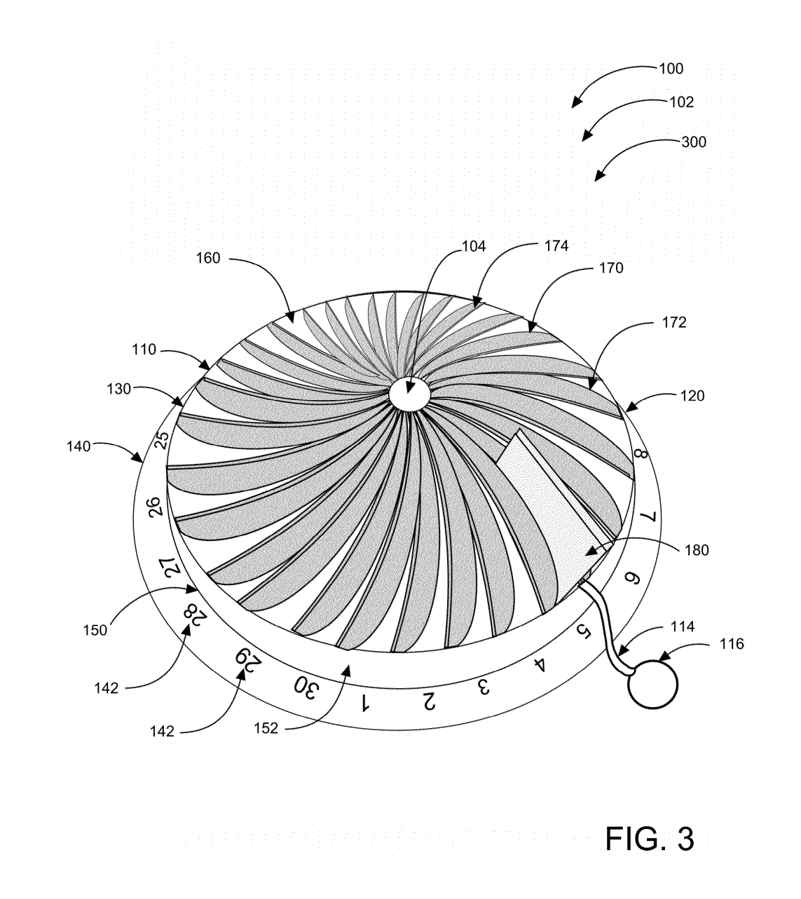

Cylindrical rim 140 further comprises plurality of numbers 142 corresponding to each compartment separated by turbine-shaped-arcuate fins 174. Plurality of numbers 142 may range from 1 through 15 to accommodate up to 15 separate compartments for independently housing up to 15 said surgical pads 180 in small version 200 of receptacle for storing and counting surgical pads assembly 102 as shown in FIG. 2. Plurality of inner fins 170 preferably comprises 15 numbers to match each separate compartment and the corresponding number located on cylindrical rim 140. In larger version 300 of receptacle for storing and counting surgical pads assembly 102 plurality of numbers 142 may range from 1 through 30 to accommodate up to 30 separate compartments 300 for housing up to 30 surgical pads 180 as shown in FIG. 3. Plurality of inner fins 170 preferably comprises 30 numbers to match each separate compartment and the corresponding number located on cylindrical rim 140.

Plurality of inner fins 170 comprises a different color as a color-coding means to signal to medical professional 108 that surgical pad 180, if inner fin 170 is visible, has not been replaced and is still inside of user-patient 190 as shown in FIG. 1. Plurality of inner fins 170 comprise concave profile 172 relative to center radius 104 and cylindrical rim 140 having the corresponding number (plurality of numbers 142). Plurality of inner fins 170 are dimensioned to be pie-shaped so as to fit inside cylindrical receptacle 110 while accommodating at least one surgical pad 190. Receptacle for storing and counting surgical pads assembly 102 may further comprises pull-string cord 114 for rotating cylindrical receptacle 110 to a different separate compartment. Pull-string cord 114 may comprise finger-engageable ring 116 to facilitate rotating of cylindrical receptacle 110.

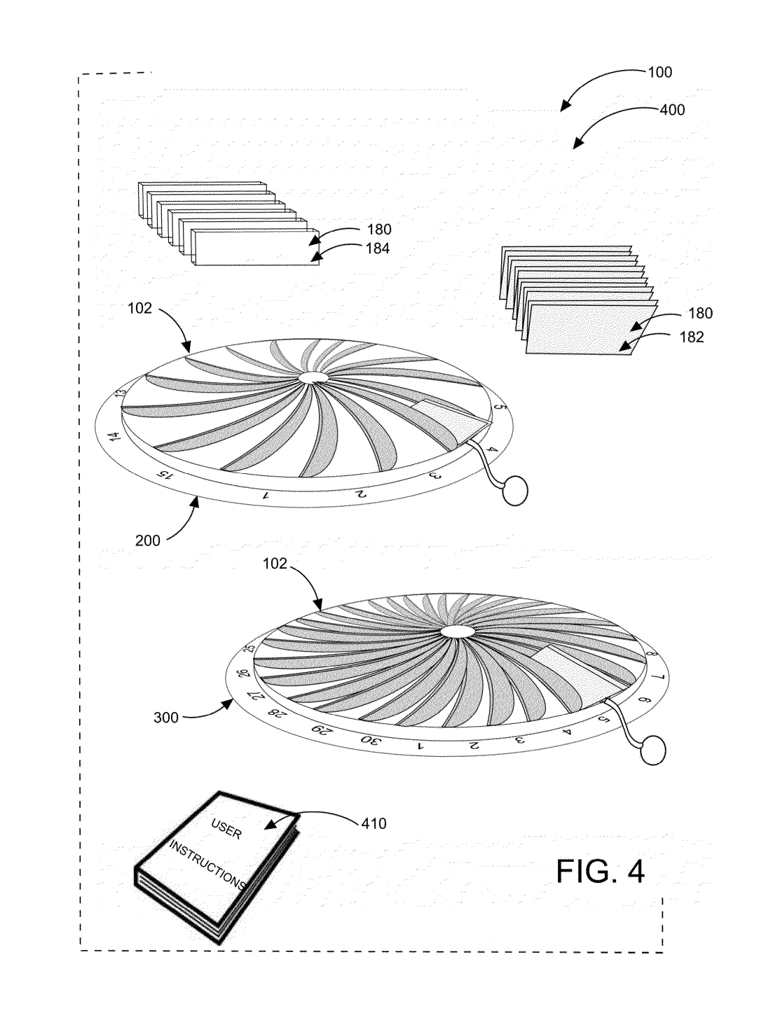

Referring now to FIG. 4 illustrating a perspective view illustrating kit 400 of receptacle for storing and counting surgical pads system 100 according to an embodiment of the present invention.

Receptacle for storing and counting surgical pads system 100 may be sold as a kit 400 comprising the following parts: plurality of receptacle for storing and counting surgical pads assemblies 102 in different sizes; plurality of surgical pads 180 including gauze pads 182 and surgical sponges 184; and at least one set of user instructions 410. Kit 400 has instructions such that functional relationships are detailed in relation to the structure of the invention (such that the invention can be used, maintained, or the like in a preferred manner). Receptacle for storing and counting surgical pads system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

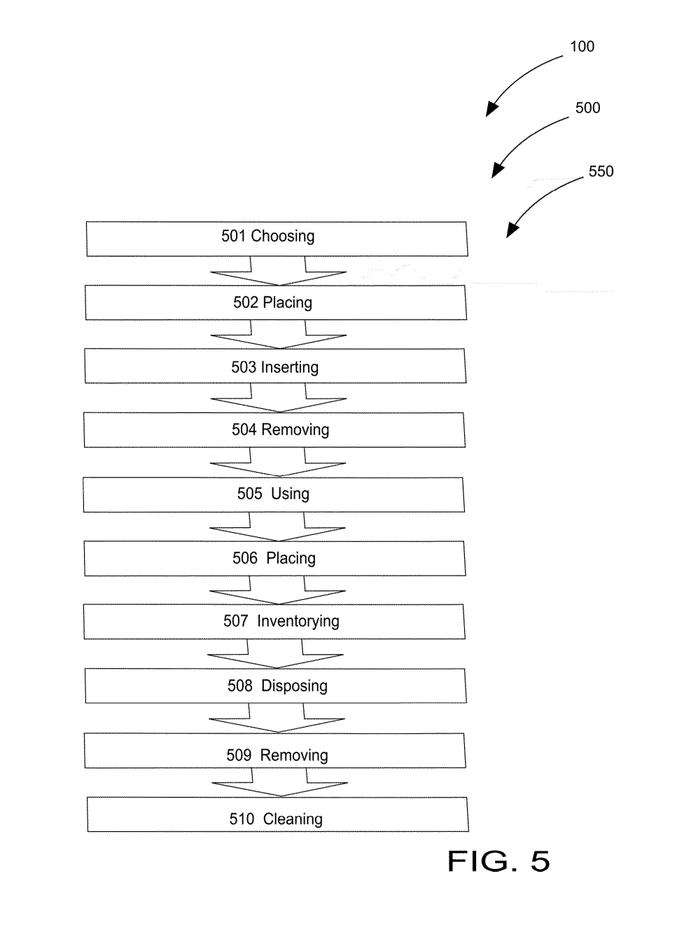

Referring now to FIG. 5, flowchart 550 illustrating method of use 500 for receptacle for storing and counting surgical pads system 100 according to an embodiment of the present invention of FIGS. 1-4.

Method of using (method of use 500) receptacle for storing and counting surgical pads system 100 comprises the steps of: step one 501 choosing receptacle for storing and counting surgical pads assembly 102 in a user-preferred size; step two 502 placing receptacle for storing and counting surgical pads assembly 102 on a surgical table 108; step three 503 inserting plurality of unused surgical pads 100 into separate numbered compartments; step four 504 removing surgical pad(s) 180 as needed during a medical procedure including surgery; step five 505 using surgical pad 180 to absorb bodily fluids of user-patient 190 undergoing medical procedure; step six 506 placing used surgical pad 180, once removed, back into separate numbered compartment (small version 200 or large version 300); step seven 507 inventorying both plurality of used surgical pads 180 and plurality of unused surgical pads 180 once the medical procedure is complete to make sure all surgical pads 180 are accounted for; step eight 508 disposing of all used surgical pads 180 in a receptacle; step nine 509 removing un-used surgical pads 180; and step ten 510 cleaning receptacle for storing and counting surgical pads assembly 102 for future use.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A receptacle for storing and counting surgical pads system comprising:
a receptacle for storing and counting surgical pads assembly including,
a cylindrical receptacle having,
a flat base;
a side wall having a cylindrical rim around a perimeter; and
a convex top section having a plurality of inner fins;
wherein said receptacle for storing and counting surgical pads system comprises said receptacle for storing and counting surgical pads assembly;
wherein said flat base, said side wall, and said convex top section comprise said cylindrical receptacle;
wherein said plurality of inner fins comprise turbine-shaped-arcuate fins extruding from a center radius;
wherein said turbine-shaped-arcuate fins are structured and arranged to provide separate adjacent compartments for storing a plurality of said surgical pads;
wherein said receptacle for storing and counting surgical pads assembly houses both used and unused said surgical pads suitably used in a variety of medical and surgical procedures; and
wherein said receptacle for storing and counting surgical pads assembly is structured and arranged to provide a means for handling, disposing, and keeping track of each said surgical pad effectively ensuring that said surgical pad is not left inside of a user-patient that would potentially cause ill-effects.

2. The receptacle for storing and counting surgical pads system of claim 1 wherein said surgical pad comprises a gauze pad.

3. The receptacle for storing and counting surgical pads system of claim 1 wherein said surgical pad comprises a surgical sponge.

4. The receptacle for storing and counting surgical pads system of claim 1 wherein said cylindrical rim around said perimeter comprises a lip.

5. The receptacle for storing and counting surgical pads system of claim 4 wherein said lip prevents used said surgical pad having at least one bodily fluid from escaping from said cylindrical receptacle thereby preventing potential contamination and excess contact with said at least one bodily fluid.

6. The receptacle for storing and counting surgical pads system of claim 4 wherein said cylindrical rim further comprises a plurality of numbers corresponding to each said compartment separated by said turbine-shaped-arcuate fins.

7. The receptacle for storing and counting surgical pads system of claim 6 wherein said plurality of numbers range from 1 through 15 to accommodate up to 15 said separate compartments for independently housing up to 15 said surgical pads.

8. The receptacle for storing and counting surgical pads system of claim 7 wherein said plurality of inner fins comprises 15 to match each said separate compartment and said corresponding number located on said cylindrical rim.

9. The receptacle for storing and counting surgical pads system of claim 6 wherein said plurality of numbers range from 1 through 30 to accommodate up to 30 said separate compartments for housing up to 30 said surgical pads.

10. The receptacle for storing and counting surgical pads system of claim 9 wherein said plurality of inner fins comprises 30 to match each said separate compartment and said corresponding number located on said cylindrical rim.

11. The receptacle for storing and counting surgical pads system of claim 6 wherein said plurality of inner fins comprises a different color as a color-coding means to signal to a medical professional that said surgical pad, if said inner fin is visible, has not been replaced and is still inside of said user-patient.

12. The receptacle for storing and counting surgical pads system of claim 11 wherein said plurality of inner fins comprise a concave profile relative to said center radius and said cylindrical rim having said corresponding number.

13. The receptacle for storing and counting surgical pads system of claim 12 wherein said plurality of inner fins are dimensioned to be pie-shaped so as to fit inside said cylindrical receptacle while accommodating said at least one surgical pad.

14. The receptacle for storing and counting surgical pads system of claim 1 wherein said flat base is able to be placed flat on a planar surface when in an in-use condition.

15. The receptacle for storing and counting surgical pads system of claim 14 wherein said planar surface comprises a surgical table.

16. The receptacle for storing and counting surgical pads system of claim 1 further comprises a pull-string cord for rotating said cylindrical receptacle to a different said separate compartment.

17. The receptacle for storing and counting surgical pads system of claim 16 wherein said pull-string cord comprises a finger-engageable ring to facilitate rotating of said cylindrical receptacle.

18. A receptacle for storing and counting surgical pads system comprising:
a receptacle for storing and counting surgical pads assembly including,
a cylindrical receptacle having,
a flat base;
a side wall having a cylindrical rim around a perimeter;
a convex top section having a plurality of inner fins; and
a pull-string cord;
wherein said receptacle for storing and counting surgical pads system comprises said receptacle for storing and counting surgical pads assembly;
wherein said flat base, said side wall, and said convex top section comprise said cylindrical receptacle;
wherein said flat base is able to be placed flat on a planar surface when in an in-use condition;
wherein said planar surface comprises a surgical table;
wherein said plurality of inner fins comprise turbine-shaped-arcuate fins extruding from a center radius;
wherein said turbine-shaped-arcuate fins are structured and arranged to provide separate adjacent compartments for storing a plurality of said surgical pads;
wherein said surgical pad comprises a gauze pad;
wherein said cylindrical rim around said perimeter comprises a lip;
wherein said lip prevents used said surgical pad having at least one bodily fluid from escaping from said cylindrical receptacle thereby preventing potential contamination and excess contact with said at least one bodily fluid;
wherein said cylindrical rim further comprises a plurality of numbers corresponding to each said compartment separated by said turbine-shaped-arcuate fins;
wherein said plurality of numbers range from 1 through 15 to accommodate up to 15 said separate compartments for independently housing up to 15 said surgical pads;
wherein said plurality of inner fins comprises 15 to match each said separate compartment and said corresponding number located on said cylindrical rim;
wherein said plurality of inner fins comprises a different color as a color-coding means to signal to a medical professional that said surgical pad, if said inner fin is visible, has not been replaced and is still inside of said user-patient;
wherein said plurality of inner fins comprise a concave profile relative to said center radius and said cylindrical rim having said corresponding number;
wherein said plurality of inner fins are dimensioned to be pie-shaped so as to fit inside said cylindrical receptacle while accommodating said at least one surgical pad;
wherein said pull-string cord for rotating said cylindrical receptacle to a different said separate compartment;
wherein said pull-string cord comprises a finger-engageable ring to facilitate rotating of said cylindrical receptacle
wherein said receptacle for storing and counting surgical pads assembly houses both used and unused said surgical pads suitably used in a variety of medical and surgical procedures; and
wherein said receptacle for storing and counting surgical pads assembly is structured and arranged to provide a means for handling, disposing, and keeping track of each said surgical pad effectively ensuring that said surgical pad is not left inside of a user-patient that would potentially cause ill-effects.

19. The receptacle for storing and counting surgical pads system of claim 17 further comprising a kit including: plurality of said receptacle for storing and counting surgical pads assembly in different sizes; a plurality of said surgical pads including gauze pads and surgical sponges; and a set of user-instructions.

20. A method of using a receptacle for storing and counting surgical pads system comprising the steps of:
choosing a receptacle for storing and counting surgical pads assembly in a user-preferred size;
placing a receptacle for storing and counting surgical pads assembly on a surgical table;
inserting a plurality of unused surgical pads into separate numbered compartments;
removing said surgical pad(s) as needed during a medical procedure including surgery;
using said surgical pad to absorb bodily fluids of a patient undergoing said medical procedure;
placing used said surgical pad, once removed, back into said separate numbered compartment;
inventorying both said plurality of used surgical pads and said plurality of unused surgical pads once said medical procedure is complete to make sure all said surgical pads are accounted for;
disposing of all used surgical pads in a receptacle;
removing un-used surgical pads; and
cleaning said receptacle for storing and counting surgical pads assembly for future use.
